(19) United States
(12) Patent Application Publication
(10) Pub. No.: US 2022/0058794 A1
(43) Pub. Date: Feb. 24, 2022

(54) PERFORMING DIAGNOSTIC ASSESSMENTS

(71) Applicant: International Business Machines Corporation, Armonk, NY (US)

(72) Inventors: Dustin Michael Sargent, San Diego, CA (US); Sun Young Park, San Diego (US)

(21) Appl. No.: 16/997,085

(22) Filed: Aug. 19, 2020

(51) Int. Cl. G06T 7/00; A61B 6/00

(52) U.S. Cl. CPC G06T 7/0012; A61B 6/502; G06T 2207/10116; G06T 2207/30068; G06T 2207/30096; A61B 6/5217

(57) ABSTRACT

Embodiments herein disclose computer-implemented methods, computer program products and computer systems for performing a diagnostic assessment. The method may include receiving image data of a current screening image, prior screening image, current diagnostic image, or prior diagnostic image, each image corresponding to a view type. The method may include processing the image data using a trained model to determine first model output data; determining first diagnosis data associated with a first confidence score; determining patch data representing a difference between the prior screening image and the current screening image; identifying a location corresponding to the difference; and determining the patch data satisfies a condition. The method may further include receiving additional image data of diagnostic images corresponding to a second type of view, processing the additional image data to determine second model output data; and determining second diagnosis data associated with a confidence score greater than the first confidence score.

### BACKGROUND

The present invention relates generally to the field of diagnostic assessments, and more particularly to classifying patient images using a deep learning model to perform a diagnostic assessment.

In performing breast cancer screening examinations on patients, a medical professional captures and reviews multiple images taken from different viewpoints and from different points in time in order to make a diagnosis. In other words, doctors use a mammogram to look for early signs of breast cancer. A mammogram is an X-ray picture of the breast. On the other hand, computer-aided diagnosis (CAD) algorithms often make decisions based on single images. Images taken during a mammogram may include different viewpoints or angles for the left breast and for the right breast. For example, standard mammogram image view types may include bilateral craniocaudal (CC), bilateral mediolateral oblique (MLO), wherein bilateral refers to the left and right versions of a view.

Typically, CAD algorithms for mammography evaluate patients based on a single study, which is assumed to contain the four standard mammography views (left CC “CC L”, left MLO “MLO L”, right CC “CC R”, and right MLO “MLO R”). These algorithms ignore disease progression that may be revealed by looking at prior exams and throw away non-standard diagnostic views that may reveal abnormalities not visible in the standard views. Such CAD algorithms and standard view only approaches do not consider how a medical professional may actually use other factors in determining a diagnosis.

### SUMMARY

The present invention is described in various embodiments disclosing methods, computer program products, and computer systems for performing a diagnostic assessment using mammograms. One embodiment of the present disclosure is a computer-implemented method for performing a diagnostic assessment, the computer-implemented method may include one or more processors configured for receiving first image data representing screening images corresponding to a first type of view, wherein the screening images comprise a prior screening image and a current screening image; processing, the first image data to determine first model output data; determining, using the first model output data, first diagnosis data corresponding to the first image data, wherein the first diagnosis data is associated with a first confidence score. The computer-implemented method may also include receiving second image data representing diagnostic images corresponding to a second type of view; processing the second image data to determine second model output data; and determining second diagnosis data associated with a second confidence score that is greater than the first confidence score, wherein the second diagnosis data is based on the first model output data and the second model output data.

Some embodiments may include one or more processors configured for determining that the first confidence score satisfies a first condition and sending an instruction to retrieve the second image data to a device. Further, the one or more processors may be configured for determining patch data representing at least a difference between the prior screening image and the current screening image and determining that the patch data satisfies a second condition. Furthermore, the one or more processors may be configured for identifying a location corresponding to the difference between the prior screening image and the current screening image, determining the second type of view based on the location, and including the location in the instruction to retrieve the second image to the device. Even furthermore, the one or more processors may be configured for determining third diagnosis data associated with a third confidence score that is greater than the second confidence score, wherein the third diagnosis data is based at least on the first model output data, the second model output data, and the patch data.

The first type of view may be one of a left craniocaudal (CC L) view, a right craniocaudal (CC R) view, a left mediolateral oblique (MLO L) view, or a right mediolateral oblique (MLO R) view. In some embodiments, the at least one screening image may include a prior screening image and a current screening image, wherein the prior screening image corresponds to the first type of view and the current screening image corresponds to the first type of view.

The computer-implemented method may further include one or more processors configured for determining, using a change detection component, patch data representing at least a difference between the prior screening image and the current screening image; identifying a location corresponding to the difference; and determining that the patch data satisfies a condition. In response to determining that the patch data satisfies the condition, the computer-implemented method may further include one or more processors configured for determining to obtain at least one image of a second type of view that includes the location and sending, to a device, an instruction to retrieve additional image data representing at least one diagnostic image corresponding to the second type of view.

The computer-implemented method may further include one or more processors configured for receiving additional image data representing at least one diagnostic image corresponding to a second type of view; processing the additional image data to determine second model output data; and determining, using the second model output data, second diagnosis data corresponding to the at least one diagnostic image, the second diagnosis data associated with a second confidence score that is greater than the first confidence score. The second type of view may be one of the following views: a mediolateral (ML) view, a lateromedial (LM) view, a lateromedial oblique (LMO) view, a late mediolateral (late ML) view, a step oblique (SO) view, a spot view, a spot compression view, a double spot compression view, a magnificent view, an exaggerated craniocaudal view (XCC), an axillary view, a cleavage view, a tangential view, a caudocranial view, a bullseye CC view, a rolled CC view, an elevated CC view, a 20° oblique view, an inferomedial superolateral oblique projection view, and an Eklund technique view. Some views of the second type of view may correspond to a medial portion or a lateral portion of the breast, indicated by an “M” for medial or an “L” for lateral (e.g., XCCM—medial, XCCL—lateral).

### BRIEF DESCRIPTION OF THE DRAWINGS

FIG. 1A depicts a flow chart of the steps of a computer-implemented method 100A for performing a diagnostic assessment, in accordance with an embodiment of the present invention.

FIG. 1B depicts a block diagram of a distributed data processing environment 100B for performing a diagnostic assessment, in accordance with an embodiment of the present invention.

FIG. 2 depicts a block diagram of a system for performing a diagnostic assessment, in accordance with an embodiment of the present invention.

FIG. 3 depicts a block diagram of a study level classifier for performing a diagnostic assessment, in accordance with an embodiment of the present invention.

FIG. 4 depicts a flow chart of a change detection model used for performing a diagnostic assessment, in accordance with an embodiment of the present invention.

FIG. 5 depicts a flow chart of a study level model used for performing a diagnostic assessment, in accordance with an embodiment of the present invention.

FIG. 6 depicts a flow chart of another study level model used for performing a diagnostic assessment, in accordance with an embodiment of the present invention.

FIG. 7 depicts a flow chart of steps of a computer-implemented method for performing a diagnostic assessment, in accordance with an embodiment of the present invention.

FIG. 8 depicts a flow chart of steps of another computer-implemented method for performing a diagnostic assessment, in accordance with an embodiment of the present invention.

FIG. 9 depicts a block diagram of a computing device of the distributed data processing environment of FIG. 1, in accordance with an embodiment of the present invention.

### DETAILED DESCRIPTION

The present invention addresses the problem of computer-aided diagnosis (CAD) algorithms making a diagnosis decision using only a single image. When mammographers screen patients for breast cancer, they review multiple images taken from different viewpoints and one or more prior exams before arriving at their diagnosis. On the other hand, CAD algorithms often make their decisions based on single images. In the best cases, CAD algorithms for mammography evaluate patients based on a single study (e.g., breast cancer examination during a patient visit), which is assumed to contain the four standard mammography views (CC L, MLO L, CC R, and MLO R). These algorithms ignore disease progression that may be revealed by looking at prior exams and discard non-standard diagnostic views that may reveal abnormalities not visible in the standard views.

The present invention therefore provides a patient-level deep learning model for breast cancer screening that solves the problems with existing CAD algorithms. During implementation, a patient may request a breast cancer screening as a routine medical examination. During the screening exam, a medical professional may capture a series of mammograms (i.e., X-ray images) to conduct the examination, wherein the captured mammograms may be used in a breast cancer diagnostic study to determine the patient's breast cancer diagnosis. A standard set of mammograms may be captured for screening purposes, and if an abnormality is detected in the standard set of mammograms, then a diagnostic set of mammograms including additional non-standard types of views may be captured to further examine the detected abnormality.

Each image taken during a mammogram includes a corresponding type of view. For example, a type of view for an image may include a standard type of view or a non-standard type of view for mammograms, in which the standard types of view are generally captured during a screening exam and the non-standard types of views are generally captured in a follow-up diagnostic exam. A standard type of view may be captured during a screening exam and may include a bilateral CC view or a bilateral MLO view, wherein a bilateral type of view may include a positional variant representing the left or right position, indicated as CC L view, CC R view, MLO L view and MLO R view. A non-standard type of view may be captured during a diagnostic exam and may include one of a mediolateral (ML) view, a lateromedial (LM) view, a lateromedial oblique (LMO) view, a late mediolateral (late ML) view, a step oblique (SO) view, a spot view, a spot compression view, a double spot compression view, a magnificent view, an exaggerated craniocaudal view (XCC), an axillary view, a cleavage view, a tangential view, a CC view, a bullseye CC view, a rolled CC view, an elevated CC view, a 20° oblique view, an inferomedial superolateral oblique projection view, and an Eklund technique view. The non-standard images may correspond to the standard images such that the non-standard images may be a variation of one or more of the standard images.

Embodiments of the present invention provide a system and method for processing mammogram image data from a standard set of images and/or a diagnostic set of images to determine a diagnostic assessment using a study-level image classifier, a patient-level change detection component, and a diagnosis model.

Embodiments of the present invention provide a study-level image classifier for processing a standard set and/or diagnostic set of mammograms to determine a classification (e.g., benign, malignant, normal) for each image in the study and produce model output data as a feature vector for each image. A study-level image classifier may include a deep convolutional neural network (“CNN”) for image classification, which may be a rule-based machine learning model that combines a discovery component (e.g., genetic algorithm) with a learning component (e.g., supervised learning or unsupervised learning). In general, image classifier systems seek to identify a set of context-dependent rules that collectively store and apply knowledge in a piecewise manner in order to make predictions (e.g., behavior modeling, classification, data mining, regression, function approximation, or game strategy). The study-level image classifier may also include a combination network component configured to concatenate the model output data feature vectors to produce a study-level classification through a series of fully connected layers. The study-level image classifier may be trained using other images tagged with medical labels or associated with annotations and/or diagnostic reports to improve the functionality and accuracy of the image classifier.

In another embodiment, the study-level image classifier may include two separate image level classifiers, wherein a first image level classifier receives two CC (i.e., CC L and CC R) images and the second image level classifier receives two MLO (i.e., MLO L and MLO R) images. Further, the study-level image classifier may include an image level classifier for each one of the four input images.

Embodiments of the present invention may further include an image mapper or image handler configured to select diagnostically relevant images from a standard set and/or diagnostic set of images for input into the study-level image model. Diagnostically relevant images may be selected by determining which images and their corresponding view types should be selected along with their prior counterparts in order to perform a diagnosis. For example, exaggerated CCM (“XCCM”) and exaggerated CCL (“XCCL”) views may be substituted for CC views, and ML and LM views may be substituted for MLO views, wherein an “M” suffixed to a view abbreviation corresponds to a medial type of the view and an “L” suffixed to a view abbreviation corresponds to a lateral type of view. The image mapper may also be configured to select other non-standard views for prior images and to synthesize those prior images using Generative Adversarial Networks (GANs). Further, optimal view combinations may be learned during model training.

Embodiments of the present invention may further be configured to include prior images as inputs to the system by stacking the prior images for each view as multi-channel images without registration. Registration may include aligning a first image to a second image so that the pixels from the first image align with the respective pixels of the second image. The system may be configured to output a prediction for the entire study, wherein the prediction may be representative of a benign or a malignant diagnosis. The system may also be configured to output a prediction for the left breast or the right breast. The system may also be configured to sort the results according to the Breast Imaging Reporting and Data System (BI-RADS), which sorts the results into categories numbered zero (0) through six (6). The system may also be configured to process the image data for each side separately and then combine the results through average or max operations. An image classifier of the system may be pre-trained to produce per-image diagnosis to improve training speed.

Further, embodiments of the present invention provide a patient-level change detection component that may be a deep learning model configured to analyze image patches between current and prior exams and detect or identify if a clinically relevant change has occurred. The change detection component may be configured to detect and identify significant changes between the standard set and/or diagnostic set of mammograms from prior and/or current mammograms.

Further, embodiments of the present invention provide a diagnosis model that produces the overall diagnosis for the patient based on output data from the study-level image classifier and the patient-level change detection component. Further, the diagnosis model may be configured to automatically detect change regions and classify the change regions according to diagnostic relevance without having learned any specific objects to detect.

Embodiments of the present invention provide a computer-implemented method for performing a diagnostic assessment that may include receiving image data representing at least one screening image corresponding to a first type of view; processing, using a trained model, the image data to determine first model output data; and determining, using the first model output data, first diagnosis data corresponding to the at least one screening image, the first diagnosis data associated with a first confidence score. The first type of view may include at least one of: a CC L view, a CC R view, a MLO L view, and a MLO R view. The at least one screening image may include a prior screening image and a current screening image, wherein the prior screening image may correspond to the first type of view and the current screening image may correspond to the first type of view.

The computer-implemented method may include determining, using a change detection component, patch data representing at least a difference between the prior screening image and the current screening image; identifying a location corresponding to the difference; and determining that the patch data satisfies a condition. For example, the condition may correspond to a binary result indicating that the patch data exceeds a predetermined threshold. For example, if a difference is determined to exist between the prior screening image and the current screening image, and that difference is greater than 2% of the entire image, then the patch data may satisfy the condition that the difference is greater than the 2% threshold. The condition may correspond to the patch data exceeding a predetermined threshold indicating an abnormality in at least one of the prior screening images and the current screening image. Other threshold methods may be implemented. The computer-implemented method may also include, in response to determining that the patch data satisfies the condition, determining to obtain at least one image of a second type of view that includes the location; and sending, to a device, an instruction to retrieve additional image data representing at least one diagnostic image corresponding to the second type of view.

In another embodiment, the computer-implemented method may include receiving additional image data representing at least one diagnostic image corresponding to a second type of view; processing the additional image data to determine second model output data; and determining, using the second model output data, second diagnosis data corresponding to the at least one diagnostic image, wherein the second diagnosis data is associated with a second confidence score that is greater than the first confidence score. The second type of view may be a non-standard type of view, e.g., a mediolateral (ML) view, a lateromedial (LM) view, a lateromedial oblique (LMO) view, a late mediolateral (late ML) view, a step oblique (SO) view, a spot view, a spot compression view, a double spot compression view, a magnificent view, an exaggerated CC view, an axillary view, a cleavage view, a tangential view, a caudocranial view, a bullseye CC view, a rolled CC view, an elevated CC view, a 20° oblique view, an inferomedial superolateral oblique projection view, and an Eklund technique view.

The present invention will now be described in detail with reference to the Figures.

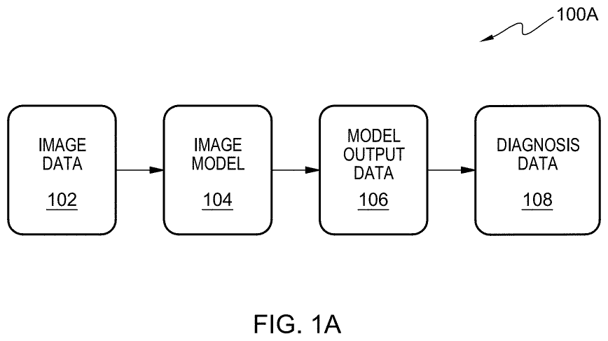

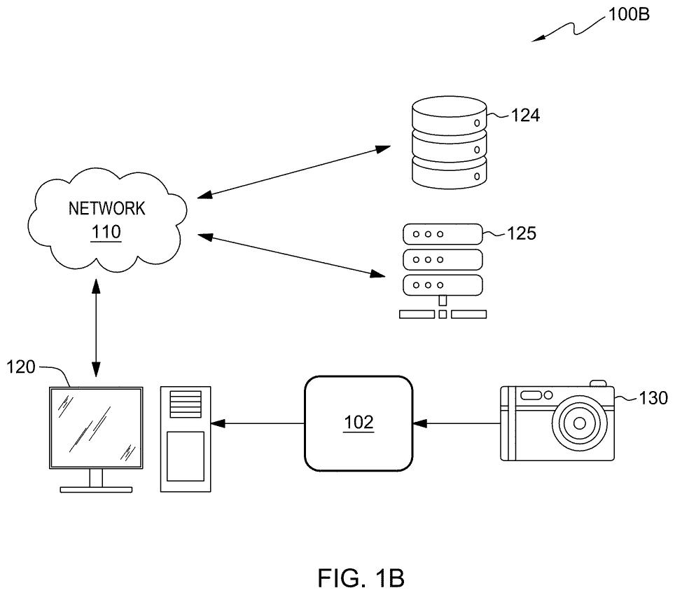

FIG. 1A depicts a flow chart of the steps of a computer-implemented method 100A for performing a diagnostic assessment, in accordance with an embodiment of the present invention. FIG. 1A provides only an illustration of one embodiment of the present invention and does not imply any limitations with regard to the environments in which different embodiments may be implemented. In the depicted embodiment, computer-implemented method 100A initiates upon receiving image data 102 at image model 104, which when processed by image model 104, generates model output data 106 used to generate diagnosis data 108 which indicates a particular diagnosis.

Image data 102 may include data corresponding to one or more images captured by a device. For example, the one or more images may include X-ray images taken during a mammogram for a breast cancer screening (i.e., patient screening images) or for a breast cancer diagnostic exam (i.e., patient diagnostic images). The one or more images may be images captured during a current screening, a prior screening, a current diagnostic exam, or a prior diagnostic exam.

As mentioned above herein, each image may include a corresponding type of view included in the image data 102. For example, a type of view for an image may include a standard type of view or a non-standard type of view for mammograms. A standard type of view may be captured during a screening exam and may include a bilateral CC view or a bilateral MLO view. A non-standard type of view may be captured during a diagnostic exam and may include one of a mediolateral (ML) view, a lateromedial (LM) view, a lateromedial oblique (LMO) view, a late mediolateral (late ML) view, a step oblique (SO) view, a spot view, a spot compression view, a double spot compression view, a magnificent view, an exaggerated craniocaudal view, an axillary view, a cleavage view, a tangential view, a caudocranial view, a bullseye CC view, a rolled CC view, an elevated CC view, a 20° oblique view, an inferomedial superolateral oblique projection view, and an Eklund technique view.

Image model 104 may be a deep learning model that may be configured to function as an image feature model or an image classifier. The deep learning model may be configured as a feature model to include a supervised or unsupervised machine learning model configured to receive input data (e.g., image data) and generate output data (e.g., a statistical value) representing the features of the input data. Image model 104 may include a supervised machine learning model (e.g., regression, classification) configured to learn a function that maps an input to an output based on example input-output pairs. In the case where image model 104 is a regression model, the regression model may be a neural network with multiple input variables and one or more output variables where the output is continuous. The input variables and output variables may be feature vectors. In the case where image model 104 is a classification model, the classification model differs from the regression model because the output is discrete. Image model 104 may include an unsupervised machine learning model (e.g., clustering, dimensionality reduction) configured to draw inferences and find patterns from input data without references to labeled outcomes. For example, input-output pairs may include an image or set of images including a malignant tumor as the input and a classification to identifying the image or set of images as a positive breast cancer diagnosis as the output. The output may be a probabilistic classifier corresponding to a confidence value that the image or set of images includes a malignant tumor. The image or set of images may include no tumors or a benign tumor as the input to image model 104, and the corresponding classification may identify the image or set of images as a negative breast cancer diagnosis or negative but benign diagnosis as the output, respectively.

Model output data 106 may include data corresponding to features or a classification of the image data 102 input to image model 104. For example, if image model 104 is a regression model and the input to image model 104 is an image, then model output data 106 may include a feature vector corresponding to the image. If image model 104 is configured to receive a certain number of inputs, model output data 106 may include the same certain number of outputs corresponding to the certain number of inputs.

Model output data 106 may be manipulated to be reduced in size for further processing. For example, a global average pooling algorithm may be applied to model output data 106 to obtain an average of all the feature vectors represented in model output data 106. Further, model output data 106 may be concatenated, which when combined with the average obtained by the global average pooling algorithm, reduces the size of the feature vectors to a single feature vector with all the data of the individual vectors represented in the single combined feature vector. Further, a dense algorithm may be used to further reduce the model output data 106 (e.g., single combined feature vector) from a first dimensional format to a second dimensional format that is compatible with a statistical algorithm (e.g., Softmax).

Diagnosis data 108 may include data corresponding to a numerical value representing the probability distributions of a list of potential outcomes. For example, diagnosis data 108 may include the outcome of a Softmax function that receives logits scores and turns the logits scores into probabilities, in which the probabilities sum to 1. Logits are the raw scores output by the last layer of a neural network. The Softmax function turn logits (i.e., the numeric output of the last linear layer of a multi-class classification neural network) into probabilities by taking the exponents of each output and then normalizing each number by the sum of those exponents, so the entire output vector or model output data adds up to 1.

FIG. 1B depicts a block diagram of a distributed data processing environment 100B for performing a diagnostic assessment, in accordance with an embodiment of the present invention. FIG. 1 provides only an illustration of one embodiment of the present invention and does not imply any limitations with regard to the environments in which different embodiments may be implemented. In the depicted embodiment, distributed data processing environment 100 includes computing device 120, server 125, database 124, and image sensor 130 interconnected over network 110. Network 110 operates as a computing network that can be, for example, a local area network (LAN), a wide area network (WAN), or a combination of the two, and can include wired, wireless, or fiber optic connections. In general, network 110 can be any combination of connections and protocols that will support communications between computing device 120, server 125, database 124, and image sensor 130. Distributed data processing environment 100 may also include additional servers, computers, sensors, or other devices not shown.

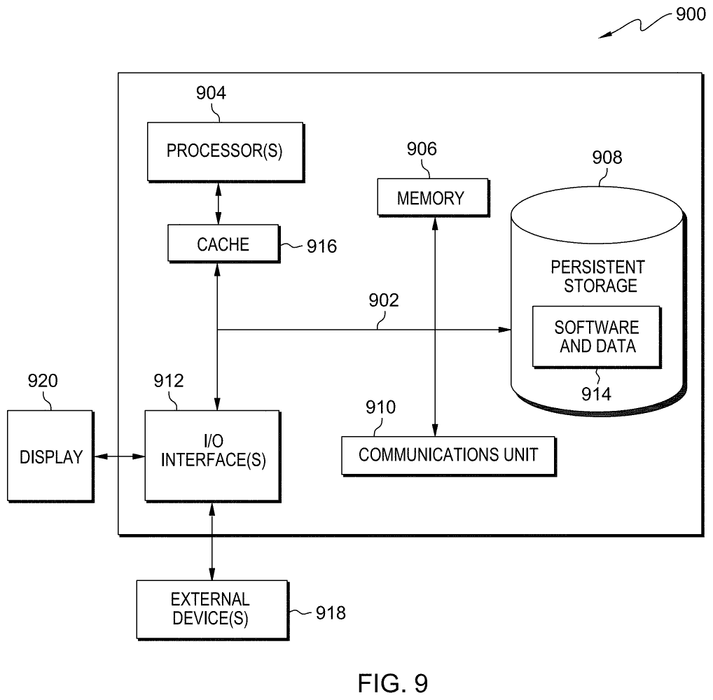

Computing device 120 operates to execute at least a part of a computer program for performing a diagnostic assessment. In an embodiment, computing device 120 may be communicatively coupled with image sensor 130 or image sensor 130 may be one of computing device 120 components. Computing device 120 be configured to send and/or receive data from network 110 and image sensor 130. In some embodiments, computing device 120 may be a management server, a web server, or any other electronic device or computing system capable of receiving and sending data. In some embodiments, computing device 120 may be a laptop computer, tablet computer, netbook computer, personal computer (PC), a desktop computer, a smart phone, or any programmable electronic device capable of communicating with database(s) 124, server(s) 125 via network 110. Computing device 120 may include components as described in further detail in FIG. 9.

Computing device 120 may also be configured to receive, store, and process images captured on image sensor 130. For example, computing device 120 may be communicatively coupled to image sensor 130 and receive, via a communications link, data corresponding to images captured by image sensor 130. Computing device 120 may be configured to store the image data in memory of computing device 120 or transmit the image data to database 124 or server 125 via network 110. The image data may be processed by one or more processors of computing device 120 or by one or more processors associated with server(s) 125 in a cloud computing network.

Database 124 operates as a repository for data flowing to and from network 110. Examples of data include user data, device data, network data, and data corresponding to images captured by image sensor 130. A database is an organized collection of data. Database 124 can be implemented with any type of storage device capable of storing data and configuration files that can be accessed and utilized by computing device 120, such as a database server, a hard disk drive, or a flash memory. In an embodiment, database 124 is accessed by computing device 120 to store data corresponding to images captured by image sensor. In another embodiment, database 124 is accessed by computing device 120 to access user data, device data, network data, and data corresponding to images captured by image sensor 130. In another embodiment, database 124 may reside elsewhere within distributed network environment 100 provided database 124 has access to network 110.

Server 125 can be a standalone computing device, a management server, a web server, or any other electronic device or computing system capable of receiving, sending, and processing data and capable of communicating with computing device 120 via network 110. In other embodiments, server 125 represents a server computing system utilizing multiple computers as a server system, such as a cloud computing environment. In yet other embodiments, server 125 represents a computing system utilizing clustered computers and components (e.g., database server computers, application server computers, etc.) that act as a single pool of seamless resources when accessed within distributed data processing environment 100. Server 125 may include components as described in further detail in FIG. 9.

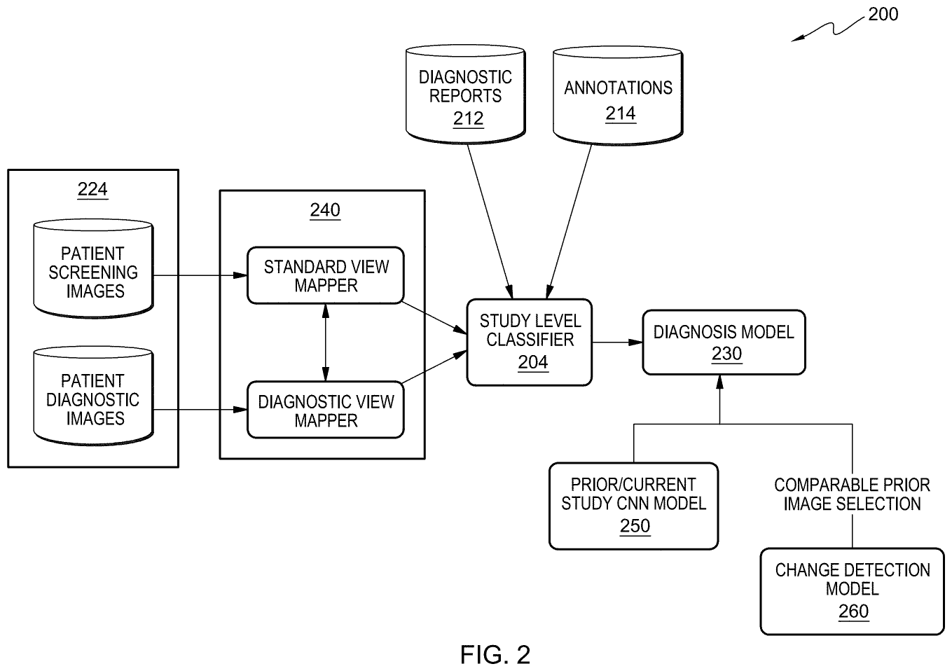

While the foregoing describes, and FIG. 2 illustrates, implementation of a study-level image model 200, the present disclosure is not limited thereto. In at least some embodiments, the model 200 may implement a trained component or trained model configured to perform the processes described above with respect to the study-level image model 200. The trained component may include one or more machine learning models, including but not limited to, one or more classifiers, one or more neural networks, one or more probabilistic graphs, one or more decision trees, and others. In other embodiments, the trained component may include a rules-based engine, one or more statistical-based algorithms, one or more mapping functions or other types of functions/algorithms to determine whether a natural language input is a complex or non-complex natural language input. In some embodiments, the trained component may be configured to perform binary classification, where the natural language input may be classified into one of two classes/categories. In some embodiments, the trained component may be configured to perform multiclass or multinomial classification, where the natural language input may be classified into one of three or more classes/categories. In some embodiments, the trained component may be configured to perform multi-label classification, where the natural language input may be associated with more than one class/category.

Various machine learning techniques may be used to train and operate trained components to perform various processes described herein. Models may be trained and operated according to various machine learning techniques. Such techniques may include, for example, neural networks (such as deep neural networks and/or recurrent neural networks), inference engines, trained classifiers, etc. Examples of trained classifiers include Support Vector Machines (SVMs), neural networks, decision trees, AdaBoost (short for “Adaptive Boosting”) combined with decision trees, and random forests. Focusing on SVM as an example, SVM is a supervised learning model with associated learning algorithms that analyze data and recognize patterns in the data, and which are commonly used for classification and regression analysis. Given a set of training examples, each marked as belonging to one of two categories, an SVM training algorithm builds a model that assigns new examples into one category or the other, making it a non-probabilistic binary linear classifier. More complex SVM models may be built with the training set identifying more than two categories, with the SVM determining which category is most similar to input data. An SVM model may be mapped so that the examples of the separate categories are divided by clear gaps. New examples are then mapped into that same space and predicted to belong to a category based on which side of the gaps they fall on. Classifiers may issue a “score” indicating which category the data most closely matches. The score may provide an indication of how closely the data matches the category.

In order to apply the machine learning techniques, the machine learning processes themselves need to be trained. Training a machine learning component requires establishing a “ground truth” for the training examples. In machine learning, the term “ground truth” refers to the accuracy of a training set's classification for supervised learning techniques. Various techniques may be used to train the models including backpropagation, statistical learning, supervised learning, semi-supervised learning, stochastic learning, or other known techniques.

FIG. 2 depicts an architecture diagram of system 200 for performing a diagnostic assessment, in accordance with an embodiment of the present invention. The system 200 may include a study level classifier 204 configured to receive image data from an image database 224. Prior to receiving the image data, an image view mapper 240 may be configured to receive the image data and determine which images should be selected for processing by study level classifier 204, and then transmit the image data corresponding to the selected images to study level classifier 204. The study level classifier 204 may be configured to receive multiple input images and produce model output data as feature vectors corresponding to each image and transmit the model output data to a series of fully connected layers. The output data from the fully connected layers may be representative of a classification for a full study. In other words, if the study is a breast cancer diagnosis study for a specific patient, model output data for the study may be a classification that is one of either a normal, a benign, or a malignant diagnosis for the patient. This model output data may be in the form of a confidence score or probability for each classification representing the likelihood of a diagnosis corresponding to a normal, benign, or malignant classification.

In an embodiment, system 200 may include image database 224 configured to store, receive, and provide image data to and from components within system 200. For example, image database 224 may include patient screening images and patient diagnostic images captured by image sensor 130 via computing device 120, in which image database 224 correlates to database 124 in FIG. 1. Patient screening images may include images captured during a patient screening exam and patient diagnostic images may include images captured during a patient diagnostic exam.

In an embodiment system 200 may include image view mapper 240 configured to determine which images are most relevant for the study and configured to replace the standard view images with the corresponding non-standard view images to increase the accuracy of the study results. Study results accuracy may be improved by using non-standard view images corresponding to diagnostic image views because the diagnostic image views are captured to include a location corresponding to an abnormality identified in the screening images. Therefore, the image view mapper 240 may be configured to replace the standard view images with the corresponding non-standard view images identified as including the abnormality shown in the standard view, but at an improved view showing more detail of the abnormality.

In an embodiment, study level classifier 204 may be modified to include two separate image-level classifiers, e.g., one taking two bilateral CC images and the other taking two bilateral MLO images, or the left and right images, or a separate image-level classifier for each input image. Study level classifier 204 may be configured to utilize image view mapper 240 to prioritize substituting XCCM, XCCL views for CC views, and ML and LM views for MLO views, as these non-standard views typically contain more diagnostically relevant information than the standard views.

In an embodiment, diagnostic reports 212 and annotations 214 may be provided to study level classifier 204 as part of model input data or separately on different channel inputs. Diagnostic reports 212 and annotations 214 may include data corresponding to information gathered about image data that is provided to study level classifier 204 during training. For example, images captured and stored in a database may include information relating to diagnostic reports 212 associated with those images. Continuing from the previous example, the information relating to diagnostic reports 212 may correspond to whether the image includes a tumor that is benign or malignant, or whether the image is void of any abnormalities or tumors. Further, images captured and stored in a database may include information relating to annotations 214 made by medical professionals or a computer program appending relevant diagnostic information to be associated with the image.

In another embodiment, system 200 may include prior/current study CNN model 250 configured to determine the most relevant prior images by stacking the prior images for each view as multi-channel images (without registration). In this case, the best combination of standard and non-standard views between the prior and current is selected for analysis.

In another embodiment, system 200 may include change detection model 260 configured to process each image from the current exam and register each current image to the corresponding image in the prior exam. A cost function may then be evaluated over each image, that produces a heatmap indicating the regions with the most discrepancy in appearance between the prior and current images.

Image patches and heatmaps may be extracted from the regions with the highest discrepancy. The image patches may then be fed (as multi-channel input images) into a convolutional neural network with a similar design to study level classifier 204. For example, change detection model 260 may be configured to take an image patch and heatmap from the current MLO L image and the prior MLO L image, produce a feature vector for each patch, and then combine the results through dense layers to produce an output describing if there has been a diagnostically significant change between the patches, i.e. lesion growth, appearance of a new abnormality, etc., if there is no significant change, or if the apparent changes are just registration errors. Change detection model 260 may then be configured to return a multi-class output, with possible labels including no change, new lesion, lesion growth, registration error, etc. Change detection model 260 may also be configured to be trained on pairs of patches from prior and current exams that have been labeled by radiologists as to whether a new finding has appeared or there has been disease progression, or whether the observed discrepancy is just due to a registration error.

However, change detection model 260 does not have to be trained to detect specific types of abnormalities; change detection model 260 only needs to learn to classify whether or not a diagnostically relevant change has occurred between the two patches.

In another embodiment, system 200 may include diagnosis model 230 configured to receive model output data from study level classifier 204, prior/current study CNN model 250 and change detection model 260 to produce an overall diagnosis for the study. For example, model output data from each one of study level classifier 204, prior/current study CNN model 250, and change detection model 260 may be received at diagnosis model 230 to generate diagnosis data associated with a confidence score. In an embodiment, study level classifier 204 may be configured to produce first model output data, which when processed by diagnosis model 230 produces first diagnosis data corresponding to a first confidence score. In another embodiment, prior/current study CNN model 250 may be configured to produce second model output data, which when processed by diagnosis model 230 produces second diagnosis data corresponding to a second confidence score, wherein the second confidence score is greater than the first confidence score, indicating a more accurate diagnosis. Alternatively, a second confidence score that is greater than a first confidence score may indicate a less accurate diagnosis, depending on the parameters of the study. In yet another embodiment, change detection model 260 may be configured to produce third model output data, which when processed by diagnosis model 230, produces third diagnosis data corresponding to a third confidence score. Furthermore, in another embodiment, diagnosis model 230 may be configured to receive one or more model output data from corresponding one or more models in the study, to produce a confidence score that increases in diagnostic accuracy as additional model output data is received at diagnosis model 230.

Each model in the system 200 may be configured to output a benign or malignant prediction for the whole study, a prediction for each side, or a BIRADs prediction. However, if the output for each model is processed by diagnosis model 230, then the prediction accuracy may be improved as additional model output data is provided to and processed by diagnosis model 230. Diagnosis model 230 may also operate on each side of the study separately and then combine the results for each side through average or max operations. In another embodiment, study level classifier 204 may be pre-trained to produce per-image diagnoses to improve training speed.

Diagnosis model 230 may be implemented as any type of neural network that accepts time-series inputs of arbitrary length, such as a recurrent neural network (“RNN”), a long short-term memory (“LSTM”), or temporal convolution network (“TCN”). Diagnosis model 230 may also be implemented as an ensemble classifier, such as random forest, that takes the multiple predictions produced by prior/current study CNN model 250 and change detection model 260 described above and combines the predictions into a final diagnosis for the patient.

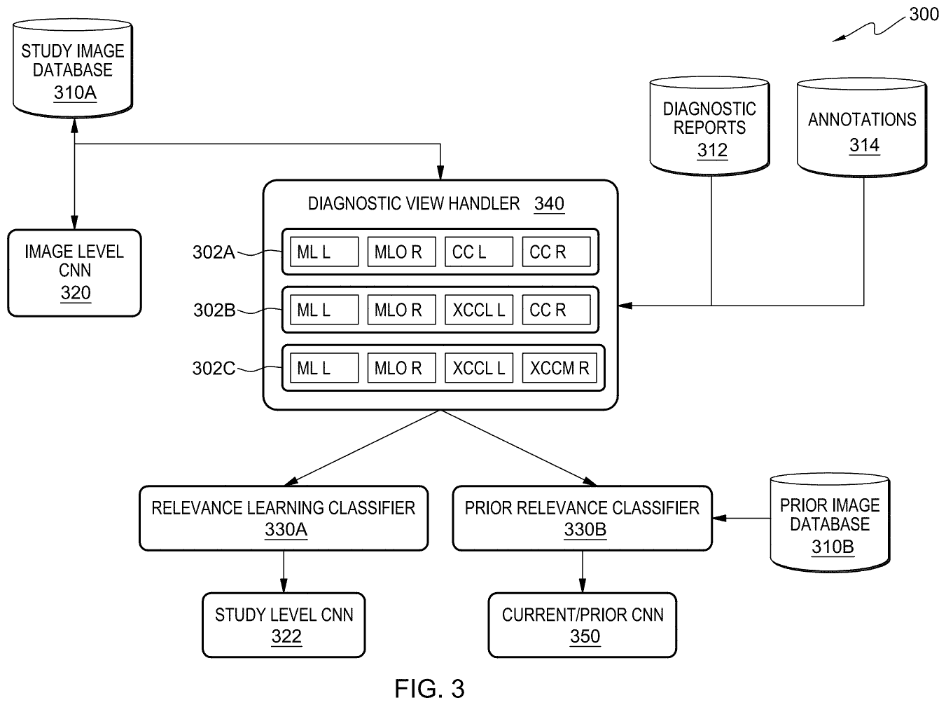

FIG. 3 depicts a block diagram of system 300 for performing a diagnostic assessment, in accordance with an embodiment of the present invention. System 300 illustrates a more detailed view of image view mapper 240 from FIG. 2 as diagnostic view handler 340 in FIG. 3.

In an embodiment, system 300 may include a diagnostic view handler 340 configured for determining which diagnostic image views to use in determining a final diagnosis, wherein the determination may be based at least on a ranking of multiple sets of images. Image data corresponding to study images stored in study image database 310A may be provided to diagnostic view handler 340. Image data may include a set of images wherein each image is of a different type of view. As shown in FIG. 3, a first set of images 302A may include ML L, MLO R, CC L and CC R view types. A second set of images 302B may include ML L, MLO R, XCCL L and CCR view types, wherein CC L from the first set of images was substituted with XCCL L for the second set of images, wherein XCCL may be an exaggerated craniocaudal lateral view and XCCL L may be an exaggerated craniocaudal lateral view of the left breast. A third set of images 302C may include ML L, MLO R, XCCL L and XCCM R view types, wherein CC R from the second set of images was substituted with XCCM R for the third set of images. XCCM may be an exaggerated craniocaudal medial view and XCCM R may be an exaggerated craniocaudal medial view of the right breast. Diagnostic view handler 340 may be configured to arrange one or more sets of images in a hierarchy wherein a first set of view may be ranked higher than a second set of view. The ranking may be based on diagnostic relevance of the set of images, wherein a set of images may be ranked highest because they show more details of a location including an abnormality.

In order to determine which set of image views should be chosen for further processing, diagnostic view handler 340 may consider data from study images stored in study image database 310A, diagnostic reports 312, and annotations 314. For example, if study image data is received from study image database 310A, and image level CNN 320 indicates that a tumor was identified in the CC L image, then diagnostic view handler 340 may be configured to substitute the CC L view image with the XCCL L view image to obtain an improved view of the identified tumor to perform a more accurate diagnosis. Similarly, any other image view type may be substituted with a corresponding image view type to obtain a more detailed look at the identified tumor and perform a more accurate diagnosis.

In another embodiment, if diagnostic view handler 340 receives data from image level CNN 320, which is stored in study image database 310A, indicating that a tumor was identified in a view type that a corresponding diagnostic image view type is not available, then diagnostic view handler 340 may be configured to send an instruction to a device to capture additional image data corresponding to the diagnostic image view type that will provide an improved view of the identified tumor. Once the additional image data is received, then diagnostic view handler 340 may be configured to substitute at least one of the current images with the additional diagnostic image data to perform a subsequent analysis with the more detailed image data or more accurate image data including more coverage of the identified tumor.

In an embodiment, study image database 310A may be a storage medium configured to store images captured by an image sensor communicatively coupled to computing device during a screening exam or a diagnostic exam, e.g., image sensor 130 and computing device 120 of FIG. 1. Prior images may be stored in prior image database 310B that may be a storage medium configured to store images captured by an image sensor communicatively coupled to computing device during a screening exam or a diagnostic exam, e.g., image sensor 130 and computing device 120 of FIG. 1.

In an embodiment, diagnostic view handler 340 may be configured to select which views of diagnostic and screening images to use in determining a diagnosis for the study. For example, three different combinations of a set of four images may be selected to use.

In an embodiment, system 300 may include one or more relevance classifiers (e.g., relevance learning classifier 330A and prior relevance classifier 330B) configured to receive model output data from diagnostic view handler 340 and process the model output data to generate classifier data for input to a CNN (e.g., study level CNN 322, current/prior CNN 350). Relevance classifiers 330A and 330B may be configured to determine which current image sets and/or prior image sets are the most diagnostically relevant images for further processing by subsequent CNN models. For example, relevance learning classifier 330A may be configured to learn that image data corresponding to current study images having an abnormality are the most relevant images to be selected for further processing by study level CNN 322. Further, prior relevance classifier 330B may be configured to determine a classification of either benign, malignant, or normal for each image to determine which of the images should be sent to current/prior CNN 350 for further processing.

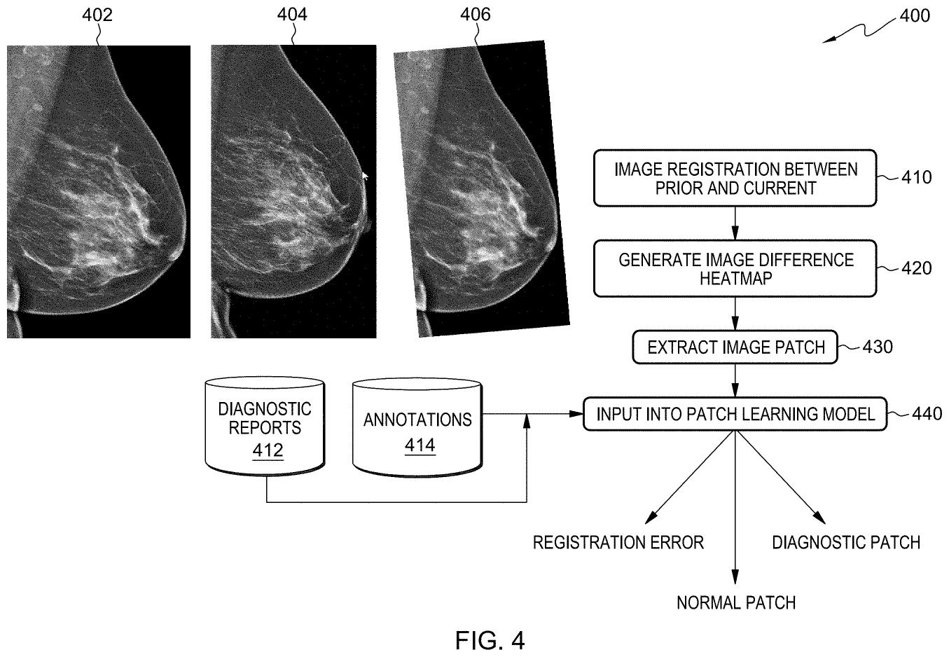

FIG. 4 depicts a flow chart of change detection model 400 used for performing a diagnostic assessment, in accordance with an embodiment of the present invention.

In an embodiment, change detection model 400 may be configured to receive image data for a current study. Image data may include, for example, a current CC L image 402, a current CC R image 404, and a prior CC L image 406. However, in order to perform a proper analysis on the current and prior images, image registration may be performed to anatomically align the current image to the prior image. For example, prior CC L image 406 may be misaligned from current CC L image 402 due to an error in capturing or uploading the image. Therefore, change detection model 400 may be configured to correct the misalignment via image registration.

In an embodiment, change detection model 400 may be configured to perform 410 image registration between a prior image and a current image, wherein the prior image is aligned with the current image to achieve anatomical congruence or anatomical alignment between the prior image and the current image. In other words, the one or more processors configured to perform 410 image registration ensures that there is a pixel to pixel match between the prior and the current image so that a proper comparison can be performed. In an embodiment, change detection model 400 may be configured to determine if the image registration was performed 410 correctly if the difference between the prior image and the current image is determined to satisfy a predetermined threshold and there are no abnormalities in the current image relative to the prior image. In other words, if there are no tumors detected in the patient images, then the difference between the current and prior images would be zero, resulting in a successful image registration between the prior image and the current image. Alternatively, if there was a tumor detected in both the prior image and the current image, then the difference between the prior image and the current image would be greater than zero if the tumor grew or changed position or shape between the two images.

However, during image registration, the difference between the prior image and the current image may be due to various reasons. First, the registration may not have been done properly and a difference may exist due to an error during the image capture or upload process. Second, the registration may not have been done properly and a difference may exist due to the patient actually having an abnormality or growth detected in the current image that was not present in the prior image. If an abnormality is detected at a location in at least one of the images, that location may be ignored in a subsequent attempt to perform 410 image registration so that the abnormality is not considered in performing a pixel-to-pixel alignment, because the registration process is triggered by determining the difference between the prior and current images is greater than zero. In other words, the abnormality may produce a difference greater than zero even though the prior image and the current image are perfectly aligned; but when the abnormality is removed, a difference of zero may be achieved.

In an embodiment, change detection model 400 may be configured to generate 420 an image difference heatmap indicating the largest discrepancies between prior images and current images. Since, registration errors will be small, the actual anatomical changes may be detected and represented as new lesions in the current image or lesions that have grown since the prior image was captured and now shown as being larger in the current image. When the maximum discrepancy regions are identified, change detection model 400 may be configured to extract 430 one or more image patches corresponding to the maximum anatomical discrepancies for both the current image and the prior image. Extracted image pairs may be input 440 into patch learning model, another CNN model, as model input data to determine if the discrepancy between the prior and current images are due to a diagnostically meaningful change or due to a registration error that is not relevant to the patient's diagnosis. For example, the patch learning model may be configured to classify the extracted image patches as a registration error, a diagnostic patch, or a normal patch. Further, patch learning model may be configured to determine if there is a diagnostically relevant change between the prior patch and the current patch. The patch learning model may be pre-trained with historical patch image data, but pre-training is not necessary to determine a diagnosis based on the patch image input data.

In an embodiment, diagnostic reports 412 and annotations 414 may be provided to patch learning model as part of model input data or separately on different channel inputs. Diagnostic reports 412 and annotations 414 may include data corresponding to information gathered about image data that is provided to patch learning model during training. For example, images captured and stored in a database may include information relating to diagnostic reports 412 associated with those images like whether the image includes a tumor that is benign or malignant, or whether the image is void of any abnormalities or tumors. Further, images captured and stored in a database may include information relating to annotations 414 made by medical professionals or a computer program appending relevant diagnostic information to be associated with the image.

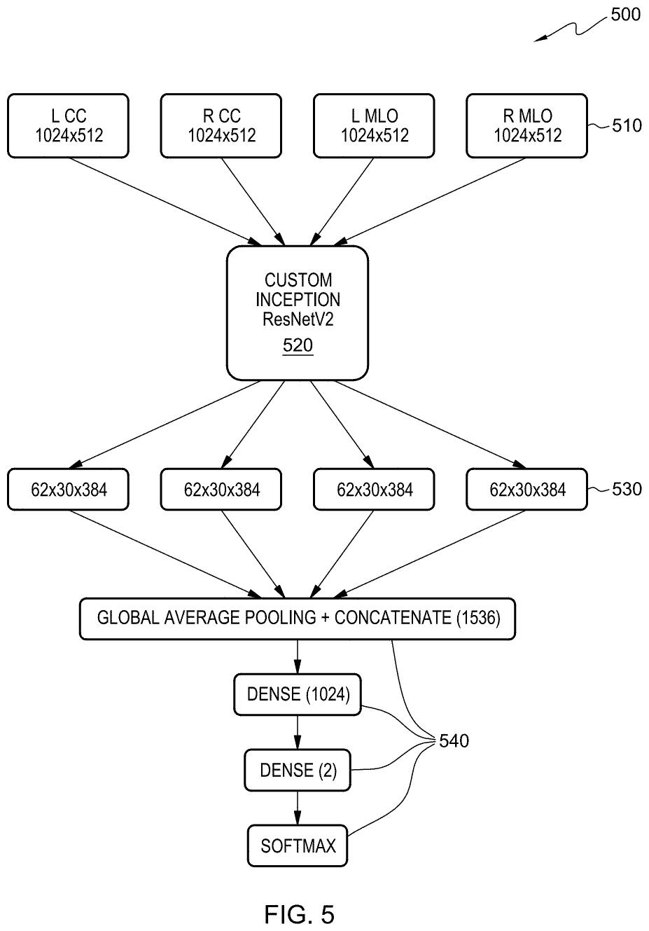

FIG. 5 depicts a process flow of study level model 500 used for performing a diagnostic assessment, in accordance with an embodiment of the present invention.

In an embodiment, study level model 500 may receive image data 510 corresponding to four standard images captured during a current patient screening exam. The four standard view images may include a CC L view, a CC R view, a MLO L view and a MLO R view, all having a pixel size of 1024×512. Image data 510 may include prior images and current images so that a difference between the current and prior images may be identified in the study. For example, the four standard view images from the current exam may be paired with four standard view images from a prior exam, wherein image data 510 may include two-channel inputs for each input to image classifier (e.g. Custom Inception ResNetV2 as depicted) 520, one channel for the current image and the other channel for the prior image, as will be described further below. In the scenario where current and prior images are included in image data 510, a current CC L view and a prior CC L view are a first two-channel input, a current CC R view and a prior CC R view are a second two-channel input, a current MLO L view and a prior MLO L view are a third two-channel input, and a current MLO R view and a prior MLO R view are a fourth two-channel input, each input having a pixel size of 1024×512×2. Thus, the image level classifier 520 may be able to simultaneously determine the difference between the current and prior images during the classification. The images may have different pixel sizes and are not limited to this example embodiment.

In an embodiment, image classifier 520 may be configured to determine the difference between current images and prior images by comparing a current image to a prior image to determine if the two images are symmetrical or asymmetrical, wherein asymmetry may indicate abnormalities and symmetry may indicate a normal result. Study level model 500 may be configured to identify a location where the asymmetry exists and determine that additional images should be captured or examined to further determine the source of the asymmetry. Asymmetry may be determined to be attributable to a poor registration (as described in FIG. 4) between two images or may be determined to be attributable to a tumor or abnormal growth in the breast tissue under examination. Poor registration may occur if at least one of the images is not properly aligned with another image under comparison. For example, as shown in FIG. 4, one image (e.g., 406) may be captured at an angle that is greater than or less than the angle of a second image (e.g., 402), wherein the angle difference exceeds a predetermined threshold.

In an embodiment, study level model 500 may receive image data 510 corresponding to non-standard images captured during a patient diagnostic exam. The non-standard images may include a ML view, a LM view, a LMO view, a late ML view, a SO view, a spot view, a spot compression view, a double spot compression view, a magnificent view, an XCC view, an axillary view, a cleavage view, a tangential view, a caudocranial view, a bullseye CC view, a rolled CC view, an elevated CC view, a 20° oblique view, an inferomedial superolateral oblique projection view, and an Eklund technique view.

In an embodiment, study level model 500 may include an image classifier (e.g., Custom Inception ResNetV2) 520 configured to process image data 510 and generate model output data 530 representing features of the image data 510. Model output data 530 may also be a 2-D feature map having 62×30×384 for each image.

In an embodiment, result combination network 540 may be configured to combine the model output data 530 to generate a binary probability. Further, result combination network 540 may be configured to performing global average pooling by taking 3-dimensional feature vectors and reduce the 3-dimensional feature vectors to one dimension by averaging the 62×30×384-dimensional feature vector for each image and concatenating the feature vectors to a one-dimensional feature vector of 1536. Result combination network 540 may also include a dense layer that is a fully connected layer where each input from the concatenated layer would connect to the fully connected layer. Softmax takes the output from the fully connected layer and generates a binary probability. A binary probability may be determined using the Softmax function, as described herein. Study level model 500 may also be configured to output a prediction for each image view provided in image data 510. Alternatively, the Softmax function may be configured to generate a 6-dimensional vector consistent with BIRADs protocols, wherein the 6-dimensional vector may include a ranking between zero (0) and six (6) depending on the severity of the diagnosis in line with the BIRADS protocols.

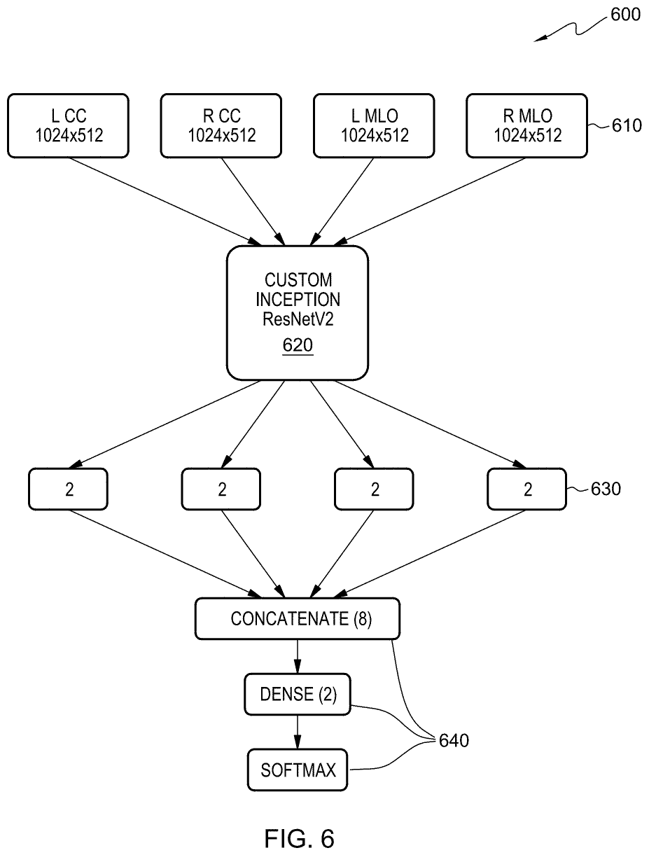

FIG. 6 depicts a process flow of study level model 600 used for performing a diagnostic assessment, in accordance with an embodiment of the present invention.

In an embodiment, study level model 600 may include an image level classifier (e.g., Custom Inception ResNetV2) 620 configured to process image data 610 and generate model output data 630 representing features of the image data 610. Model output data 630 may also be a 2-D feature map providing a classification for each image, wherein the classification may be one of various diagnoses. For example, model output data 630 may provide a classification for each image corresponding to a benign diagnosis or a malignant diagnosis. Result combination network 640 may be configured to receive model output data 630, and may then be configured to concatenate and reduce model output data 630 to a single feature vector by a dense function, which may then be provided to a Softmax function as described above.

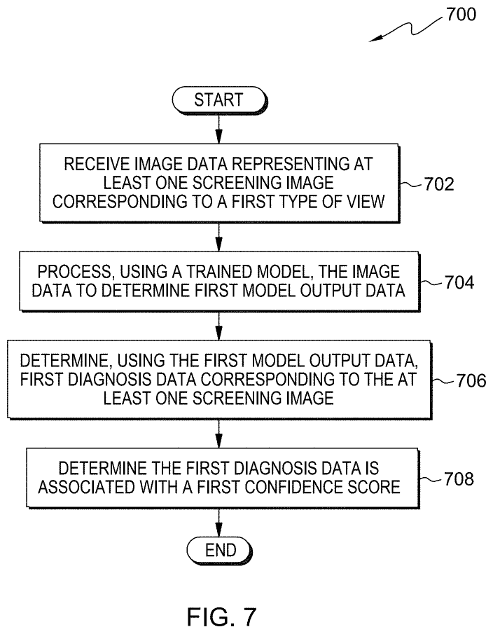

FIG. 7 depicts a flow chart of a computer-implemented method 700 for performing a diagnostic assessment, in accordance with an embodiment of the present invention.

In an embodiment, computer-implemented method 700 may include one or more processors configured to receive 702 image data representing at least one screening image corresponding to a first type of view.

Further, method 700 may include one or more processors configured to process 704, using a trained model, the image data to determine first model output data.

Further, method 700 may further include one or more processors configured to determine 706, using the first model output data, first diagnosis data corresponding to the at least one screening image, wherein the first diagnosis data may be associated with a first confidence score 708.

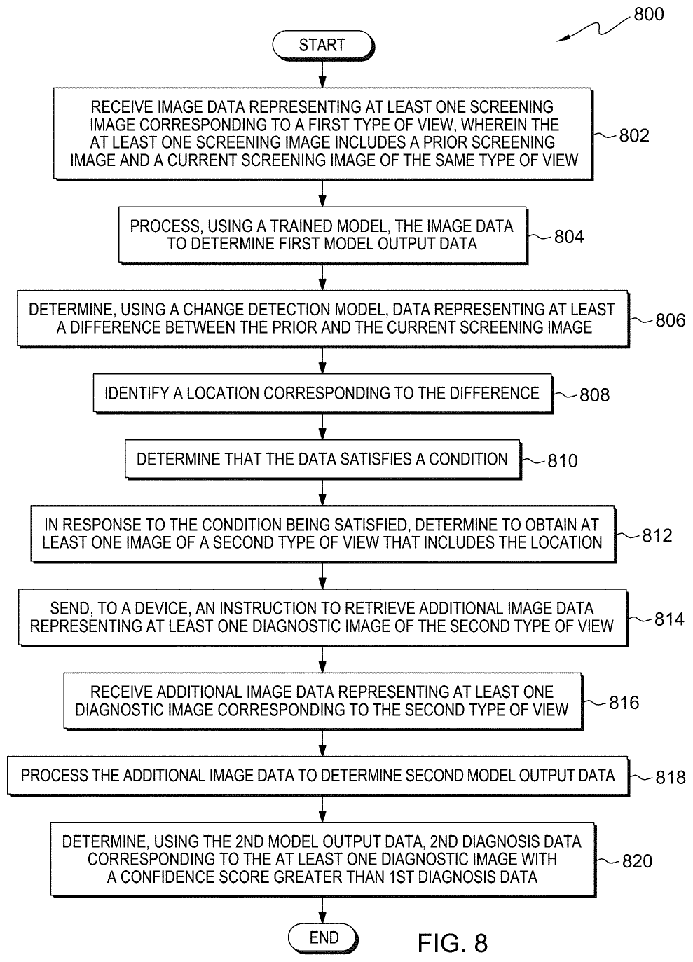

FIG. 8 depicts a flow chart of another embodiment of a computer-implemented method 800 for performing a diagnostic assessment, in accordance with an embodiment of the present invention.

In an embodiment, computer-implemented method 800 may include one or more processors configured to receive 802 image data representing at least one screening image corresponding to a first type of view, wherein the at least one screening image may include a prior screening image and a current screening image of the same type of view.

Further, computer-implemented method 800 may include one or more processors configured to process 804, using a trained model, the image data to determine first model output data.

Further, computer-implemented method 800 may further include one or more processors configured to determine 806, using a change detection model, data representing at least a difference between the prior screening image and the current screening image.

Further, computer-implemented method 800 may further include one or more processors configured to identify 808 a location corresponding to the difference between the prior screening image and the current screening image.

Further, computer-implemented method 800 may further include one or more processors configured to determine 810 that the data representing the difference between the prior screening image and the current screening image satisfies a condition.

Further, in response to the condition being satisfied, computer-implemented method 800 may further include one or more processors configured to determine 812 to obtain at least one image of a second type of view that includes the location.

Further, computer-implemented method 800 may further include one or more processors configured to send 814, to a device, an instruction to retrieve additional image data representing at least one diagnostic image of the second type of view.

Further, computer-implemented method 800 may further include one or more processors configured to receive 816 additional image data representing at least one diagnostic image corresponding to the second type of view.

Further, computer-implemented method 800 may further include one or more processors configured to process 818 the additional image data to determine second model output data.

Further, computer-implemented method 800 may further include one or more processors configured to determine 820, using the second model output data, second diagnostic data corresponding to the at least one diagnostic image with a confidence score greater than the first diagnosis data.

FIG. 9 depicts a block diagram of a computing device of distributed computing environment, in accordance with an embodiment of the present invention. FIG. 9 depicts a block diagram of computing device 900 suitable for server(s) 125 and computing device 120, in accordance with an illustrative embodiment of the present invention. It should be appreciated that FIG. 9 provides only an illustration of one implementation and does not imply any limitations with regard to the environments in which different embodiments may be implemented. Many modifications to the depicted environment may be made.

Computing device 900 includes communications fabric 902, which provides communications between cache 916, memory 906, persistent storage 908, communications unit 910, and input/output (I/O) interface(s) 912. Communications fabric 902 can be implemented with any architecture designed for passing data and/or control information between processors (such as microprocessors, communications and network processors, etc.), system memory, peripheral devices, and any other hardware components within a system. For example, communications fabric 902 can be implemented with one or more buses or a crossbar switch.

Memory 906 and persistent storage 908 are computer readable storage media. In this embodiment, memory 906 includes random access memory (RAM). In general, memory 906 can include any suitable volatile or non-volatile computer readable storage media. Cache 916 is a fast memory that enhances the performance of computer processor(s) 904 by holding recently accessed data, and data near accessed data, from memory 906.

Programs may be stored in persistent storage 908 and in memory 906 for execution and/or access by one or more of the respective computer processors 904 via cache 916. In an embodiment, persistent storage 908 includes a magnetic hard disk drive. Alternatively, or in addition to a magnetic hard disk drive, persistent storage 908 can include a solid state hard drive, a semiconductor storage device, read-only memory (ROM), erasable programmable read-only memory (EPROM), flash memory, or any other computer readable storage media that is capable of storing program instructions or digital information.

The media used by persistent storage 908 may also be removable. For example, a removable hard drive may be used for persistent storage 908. Other examples include optical and magnetic disks, thumb drives, and smart cards that are inserted into a drive for transfer onto another computer readable storage medium that is also part of persistent storage 908.

Communications unit 910, in these examples, provides for communications with other data processing systems or devices. In these examples, communications unit 910 includes one or more network interface cards. Communications unit 910 may provide communications through the use of either or both physical and wireless communications links. Programs, as described herein, may be downloaded to persistent storage 908 through communications unit 910.

I/O interface(s) 912 allows for input and output of data with other devices that may be connected to computing device 120. For example, I/O interface 912 may provide a connection to external devices 918 such as image sensor 130, a keyboard, a keypad, a touch screen, and/or some other suitable input device. External devices 918 can also include portable computer readable storage media such as, for example, thumb drives, portable optical or magnetic disks, and memory cards. Software and data 914 used to practice embodiments of the present invention can be stored on such portable computer readable storage media and can be loaded onto persistent storage 908 via I/O interface(s) 912. I/O interface(s) 912 also connect to a display 920.

Display 920 provides a mechanism to display data to a user and may be, for example, a computer monitor.

Software and data 914 described herein is identified based upon the application for which it is implemented in a specific embodiment of the invention. However, it should be appreciated that any particular program nomenclature herein is used merely for convenience, and thus the invention should not be limited to use solely in any specific application identified and/or implied by such nomenclature.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer-implemented method for performing a diagnostic assessment, comprising:
receiving, by one or more processors, first image data representing screening images each corresponding to a first type of view, wherein the screening images comprise a prior screening image and a current screening image;
processing, by the one or more processors, the first image data to determine first model output data;
determining, by the one or more processors, using the first model output data, first diagnosis data corresponding to the first image data, wherein the first diagnosis data is associated with a first confidence score;
receiving, by the one or more processors, second image data representing diagnostic images corresponding to a second type of view;
processing, by the one or more processors, the second image data to determine second model output data; and
determining, by the one or more processors, second diagnosis data associated with a second confidence score that is greater than the first confidence score, wherein the second diagnosis data is based on the first model output data and the second model output data.

2. The computer-implemented method of claim 1, further comprising:
determining, by the one or more processors, that the first confidence score satisfies a first condition; and
sending, by the one or more processors, an instruction to retrieve the second image data to a device.

3. The computer-implemented method of claim 2, further comprising:
determining, by the one or more processors, patch data representing at least a difference between the prior screening image and the current screening image; and
determining, by the one or more processors, that the patch data satisfies a second condition.

4. The computer-implemented method of claim 3, further comprising:
identifying, by the one or more processors, a location corresponding to the difference between the prior screening image and the current screening image;
determining, by the one or more processors, the second type of view based on the location; and
including, by the one or more processors, the location in the instruction to retrieve the second image data to the device.

5. The computer-implemented method of claim 3, further comprising:
determining, by the one or more processors, third diagnosis data associated with a third confidence score that is greater than the second confidence score, wherein the third diagnosis data is based on the first model output data, the second model output data and the patch data.

6. The computer-implemented method of claim 1, wherein the first type of view is at least one of: a left craniocaudal (CC L) view, a right craniocaudal (CC R) view, a left mediolateral oblique (MLO L) view, and a right mediolateral oblique (MLO R) view.

7. The computer-implemented method of claim 1, wherein the second type of view is at least one of: a mediolateral (ML) view, a lateromedial (LM) view, a lateromedial oblique (LMO) view, a late mediolateral (late ML) view, a step oblique (SO) view, a spot view, a spot compression view, a double spot compression view, a magnificent view, an exaggerated craniocaudal view, an axillary view, a cleavage view, a tangential view, a caudocranial view, a bullseye CC view, a rolled CC view, an elevated CC view, a 20° oblique view, an inferomedial superolateral oblique projection view, and an Eklund technique view.

8. The computer-implemented method of claim 1, wherein the prior screening image corresponds to the first type of view and the current screening image corresponds to the first type of view.

9. A computer program product for performing a diagnostic assessment, the computer program product comprising:
one or more computer readable storage media and program instructions stored on the one or more computer readable storage media, the program instructions comprising:
program instructions to receive first image data representing screening images each corresponding to a first type of view, wherein the screening images comprise a prior screening image and a current screening image;
program instructions to process, using a first model, the first image data to determine first model output data;
program instructions determine, using the first model output data, first diagnosis data corresponding to the first image data, wherein the first diagnosis data is associated with a first confidence score;
program instructions to receive second image data representing diagnostic images corresponding to a second type of view;
program instructions to process the second image data to determine second model output data; and
program instructions to determine second diagnosis data associated with a second confidence score that is greater than the first confidence score, wherein the second diagnosis data is based on the first model output data and the second model output data.

10. The computer program product of claim 9, further comprising:
program instructions to determine that the first confidence score satisfies a first condition; and
program instructions to send an instruction to retrieve the second image data to a device.

11. The computer program product of claim 10, further comprising:
program instructions to determine patch data representing at least a difference between the prior screening image and the current screening image; and
program instructions to determine that the patch data satisfies a second condition.

12. The computer program product of claim 11, further comprising:
program instructions to identify a location corresponding to the difference between the prior screening image and the current screening image;
program instructions to determine the second type of view based on the location; and
program instructions to include the location in the instruction to retrieve the second image data to the device.

13. The computer program product of claim 11, further comprising:
program instructions to determine third diagnosis data associated with a third confidence score that is greater than the second confidence score, wherein the third diagnosis data is based on the first model output data, the second model output data and the patch data.

14. The computer program product of claim 9, wherein the first type of view is at least one of: a left craniocaudal (CC L) view, a right craniocaudal (CC R) view, a left mediolateral oblique (MLO L) view, and a right mediolateral oblique (MLO R) view.

15. The computer program product of claim 9, wherein the second type of view is at least one of: a mediolateral (ML) view, a lateromedial (LM) view, a lateromedial oblique (LMO) view, a late mediolateral (late ML) view, a step oblique (SO) view, a spot view, a spot compression view, a double spot compression view, a magnificent view, an exaggerated craniocaudal view, an axillary view, a cleavage view, a tangential view, a caudocranial view, a bullseye CC view, a rolled CC view, an elevated CC view, a 20° oblique view, an inferomedial superolateral oblique projection view, and an Eklund technique view.

16. The computer program product of claim 9, wherein the prior screening image corresponds to the first type of view and the current screening image corresponds to the first type of view.

17. A computer system for performing a diagnostic assessment, the computer system comprising:
one or more computer processors;
one or more computer readable storage media;
program instructions stored on the one or more computer readable storage media for execution by at least one of the one or more processors, the program instructions comprising:
program instructions to receive first image data representing screening images each corresponding to a first type of view, wherein the screening images comprise a prior screening image and a current screening image;
program instructions to process, using a first model, the first image data to determine first model output data;
program instructions determine, using the first model output data, first diagnosis data corresponding to the first image data, wherein the first diagnosis data is associated with a first confidence score;
program instructions to receive second image data representing diagnostic images corresponding to a second type of view;
program instructions to process the second image data to determine second model output data; and
program instructions to determine second diagnosis data associated with a second confidence score that is greater than the first confidence score, wherein the second diagnosis data is based on the first model output data and the second model output data.

18. The computer system of claim 17, further comprising:
program instructions to determine that the first confidence score satisfies a first condition; and
program instructions to send an instruction to retrieve the second image data to a device.

19. The computer system of claim 18, further comprising:
program instructions to determine patch data representing at least a difference between the prior screening image and the current screening image; and
program instructions to determine that the patch data satisfies a second condition.

20. The computer system of claim 19, further comprising:
program instructions to identify a location corresponding to the difference between the prior screening image and the current screening image;
program instructions to determine the second type of view based on the location; and
program instructions to include the location in the instruction to retrieve the second image data to the device.

21. The computer system of claim 19, further comprising:
program instructions to determine third diagnosis data associated with a third confidence score that is greater than the second confidence score, wherein the third diagnosis data is based on the first model output data, the second model output data and the patch data.

22. The computer system of claim 17, wherein the first type of view is at least one of: a left craniocaudal (CC L) view, a right craniocaudal (CC R) view, a left mediolateral oblique (MLO L) view, and a right mediolateral oblique (MLO R) view.

23. The computer system of claim 17, wherein the second type of view is at least one of: a mediolateral (ML) view, a lateromedial (LM) view, a lateromedial oblique (LMO) view, a late mediolateral (late ML) view, a step oblique (SO) view, a spot view, a spot compression view, a double spot compression view, a magnificent view, an exaggerated craniocaudal view, an axillary view, a cleavage view, a tangential view, a caudocranial view, a bullseye CC view, a rolled CC view, an elevated CC view, a 20° oblique view, an inferomedial superolateral oblique projection view, and an Eklund technique view.

24. The computer system of claim 17, wherein the prior screening image corresponds to the first type of view and the current screening image corresponds to the first type of view.

25. The computer system of claim 19, wherein the second condition corresponds to the patch data exceeding a predetermined threshold indicating an abnormality in at least one of the prior screening images and the current screening image.
